(19) United States
(12) Patent Application Publication
(10) Pub. No.: US 2018/0092934 A1
(43) Pub. Date: Apr. 5, 2018

(54) COMPOSITION FOR DNA PROTECTION

(71) Applicant: Harsha CHIGURUPATI, Hyderabad (IN)

(72) Inventors: Manish Radheshyam BIYANI, Hyderabad (IN); Biswajit AUDDY, Hyderabad (IN); Shrabana CHAKRABARTI, Hyderabad (IN)

(21) Appl. No.: 15/394,117

(22) Filed: Dec. 29, 2016

(30) Foreign Application Priority Data
Sep. 30, 2016 (IN) 201641033595

(51) Int. Cl. A61K 31/704; A61K 31/047; A61K 9/00; C12G 3/06

(52) U.S. Cl. CPC A61K 31/704; C12G 3/06; A61K 9/0095; A61K 31/047

(57) ABSTRACT

The invention discloses a composition for DNA protection. More particularly, the invention discloses a composition to reduce DNA damage, to repair the damaged DNA and to enhance the DNA repair wherein such DNA damage may be caused due to alcohol consumption or due to any other known or unknown reasons.

### FIELD OF INVENTION

The invention relates to a composition for DNA protection. More particularly, the invention relates to a composition to reduce DNA damage and to enhance the DNA repair wherein such DNA damage may be caused due to alcohol consumption or due to any other known or unknown reasons.

### BACKGROUND OF THE INVENTION

Deoxyribo Nucleic Acid (DNA) which is an important part of the human cell constantly gets eroded or damaged by various chemicals and agents. This process is called DNA damage. This damage may be due any internal or external factor on the body and if the damaged DNA is not repaired or if not correctly repaired it may lead to mutation and serious genetic disorders or genetic instability.

### DNA Damage

DNA damage has genotoxic and cytotoxic effects on the cell. The biological consequences of the damaged cells depend upon the chemical nature of the lesion. If the lesions occur in germ cells they are heritable and will be harmful to the next generation in passing on a heritable disease. Damage in somatic cells plays an important role in cancer and aging. In order to maintain the integrity of the genome, the prokaryotic and eukaryotic organisms are well equipped with several DNA repair mechanism pathways. (Tuteja, Narendra, Mohan B. Singh, Mithilesh K. Misra, Prem L. Bhalla, and Renu Tuteja. “Molecular Mechanisms of DNA Damage and Repair: Progress in Plants.” Critical Reviews in Biochemistry and Molecular Biology 2001, 36, 4, 337-97.)

### Types and Sources of DNA Damage

All livings cells of the human body are continuously challenged as they are sensitive to spontaneous hydrolysis, which leads to DNA damage. The free radical associated with oxidatively induced DNA damage typically is 8-hydroxy-deoxyguanosine (also called 8-hydroxy-2′-deoxyguanosine). UV damage is associated with DNA single strand breaks (SSBs) or DNA double strand breaks (DSBs). Some DNA aberrations arise via physiological strand breaks caused by abortive topoisomerase I and topoisomerase II activity c) Hydrolytic DNA damage associated with Reactive Oxygen Species (ROS) involves base deamination (apurinic and apyrimidinic sites) and d) non-enzymatic methylations generate thousands of DNA-base lesions per cell per day.

Reactive oxygen species (ROS) formed continuously as a consequence of by-products from oxidative respiration or through redox-cycling events involving environmental toxic agents and Fenton reactions mediated by heavy metals leads to DNA damage. Reactive oxygen and nitrogen species are also produced by macrophages and neutrophils at sites of inflammation and infections. Such chemicals attack DNA, leading to adduct that impair base pairing and/or block DNA replication and transcription, base loss, or DNA single-strand breaks (SSBs). It is estimated that about 100-500, 8-Hydroxy-2′-deoxyguanosine (8-OHdG) lesions due to oxidative damage form per day in a human cell. The formamido pyrimidine lesions, 2, 6-diamino-4-hydroxy-5-formamidopyrimidine (FapyG) and 4,6-diamino-5-formamido pyrimidine are also formed at similar rates as 8-OH-dG after oxidative stress. (Dizdaroglu, Miral, Pawel Jaruga, Mustafa Birincioglu, and Henry Rodriguez. “Free Radical-induced Damage to DNA: Mechanisms and Measurement.” This Article Is Part of a Series of Reviews on “Oxidative DNA Damage and Repair.” Free Radical Biology and Medicine 2002, 32, 1, 1102-115.).

Apart from endogenous sources, DNA can also be damaged by exogenous agents from the environment. These include physical genotoxic stresses such as UV light from sunlight which induces a variety of mutagenic and cytotoxic DNA lesions such as cyclobutane-pyrimidine dimers (CPDs) and 6-4 photoproducts (6-4PPs).

DNA damage in the form of double strand breaks (DSBs) is incurred as a result of medical treatments like radiotherapy, ionising radiation (IR) exposure from cosmic radiation. More recently, the radiation leakage from the Fukushima power plant in Japan are examples of other sources of severe exposure to exogenous radiation.

Chemical sources of DNA damage include chemotherapeutic drugs used in cancer therapy or for other medical conditions. Alkylating agents such as methyl methanesulfonate (MMS) induce alkylation of bases, whereas drugs such as mitomycin C, cisplatin and nitrogen mustard cause DNA interstrand cross links (ICLs), and DNA intrastrand cross links. Chemotherapeutic drugs like camptothecin and etoposide are topoisomerse I and II inhibitors respectively, and gives rise to SSBs or DSBs by trapping topoisomerase-DNA complexes.

Other exogenous DNA damaging sources, that are carcinogenic as well, are foods contaminated with fungal toxins, such as the aflatoxin and overcooked meat products containing heterocyclic amines. Another common source of environmental mutagen is tobacco smoke, which generates DNA lesions in the form of aromatic adducts on DNA and SSBs also micronutrient deficiency can mimic radiation (or chemicals) in damaging DNA by SSBs or DSBs, or oxidative lesions, or both. Those micronutrients whose deficiency mimics radiation are folic acid, B₁₂, B₆, niacin, C, E, iron, and zinc. (Vaidehi Krishnan, Baohua Liu and Zhongjun Zhou. “DNA Repair, Human Diseases and Aging”, DNA Repair and Human Health, Dr. Sonya Vengrova (Ed.), 2011, ISBN: 978-953-307-612-6, InTech,). (Ames, Bruce N. “Micronutrient Deficiencies: A Major Cause of DNA Damage.” Annals NY Acad Sci Annals of the New York Academy of Sciences: 87-106.)

### DNA Damage Detection Strategies

There are number of strategies such as PCR (polymerase chain reaction), comet assay, Enzyme-linked immunosorbent assay (ELISA), Radio immunoassay (RIA), etc., that are commonly used to detect DNA damage in various organisms. (Sunita Kumari, Rajesh P. Rastogi, Kanchan L. Singh, Shailendra P. Singh and Rajeshwar P. Sinha. “DNA Damage: Detection Strategies” EXCL1 Journal 2008; 7:44-62.)

### Types of DNA Repair

There are four major classes of DNA repair pathways. They are 1. Direct repair 2. Base excision repair 3. Nucleotide excision repair and 4. Mismatch repair.

An example of direct repair is O⁶-methylguanine methyltransferase. Base excision repair pathway addresses ethenobase adducts. Nucleotide excision repair addresses malondialdehyde-dG lesion. Mismatch repair system is primarily responsible for repair of DNA replication errors. DNA replication after chronic alcohol abuse burdens the mismatch repair system.

### Ailments Caused Due to DNA Damage

Cells possess a mechanism called DNA Damage Response (DDR) to detect DNA lesions, signal their presence and promote their repair. Cells defective in these mechanisms display heightened sensitivity towards DNA damaging agents and many such defects cause human disease. (Jackson, Stephen P., and Jiri Bartek. “The DNA-damage Response In Human Biology And Disease.” Nature: 1071-078.) Different diseases like cancer, atherosclerosis, diabetes, Alzheimer's disease and sometimes serious chronic diseases including ocular disorders such as cataract, glaucoma and age-related macular degeneration (AMD) are related to changes in biomolecules like DNA.

### DNA Damage and Cancer:

Among the targets of ROS, DNA appears most important in tumor biology since it is firmly established that cancer is a genetic disease.

Oxidative mechanisms have been demonstrated to possess a potential role in the initiation, promotion, and malignant conversion (progression) stages of carcinogenesis. ROS induce several kinds of DNA damage, including strand breakage, base modification and DNA-protein cross-linkage. Supportive of this proposal are the findings that G:C to T:A transversion potentially derived from 8-Hydroxy-2′-deoxyguanosine (8-OHdG) have been observed in vivo in the ras oncogene and the p53 tumor suppressor gene in lung and liver cancer. G:C to T:A transversion is not unique to 8-OHdG, whereas G:C to T:A substitutions in the absence of UV in internal tumors has been identified as signature mutations for ROS. (Cooke, M. S. “Oxidative DNA Damage: Mechanisms, Mutation, And Disease”. The FASEB Journal 2003, 17, 10, 1195-1214.)

### Neuro Degeneration and DNA Damage

Neurodegeneration is attributed to many nervous system and aging diseases, such as Ataxia Telangiectasia (AT), Nijmegen breakage syndrome (NBS), Huntington's disease (HD), Parkinson's disease (PD) and Alzheimer's disease (AD). Furthermore, deficiency in repair of nuclear and mitochondrial DNA damage has been linked to several neurodegenerative disorders. Thus, accumulation of DNA lesions in repair-defective patients and possibly in ageing normal individuals progressively deprive neurons of vital transcripts, leading to cell dysfunction or apoptosis. (Jackson, Stephen P., and Jiri Bartek. “The DNA-damage Response In Human Biology And Disease.” Nature: 1071-078.) (Souza-Pinto, Nadja C. De, David M. Wilson, Tinna V. Stevnsner, and Vilhelm A. Bohr. “Mitochondrial DNA, Base Excision Repair and Neurodegeneration.” DNA Repair 7.7 2008, 1098-109.)

Chronic ethanol exposure increases N-methyl-D-aspartate (NMDA) receptor binding in brain cells, which increases the susceptibility of cerebellar granule cells to glutamate toxicity. Ethanol withdrawal increased neuronal excitability and neurodegenration even for low doses of NMDA. Thus NMDA plays key role in neurotoxic effects of ethanol withdrawal. The excitotoxic effects of NMDA receptor activation involves the generation of ROS. This suggests a possible role of DNA damage as consequence of ROS and lipid peroxidation due to elevated levels of CYP2E1 and/or NMDA receptor activation during chronic ethanol exposure and withdrawal.

### Ageing, Stem-Cell Dysfunction, Cardiovascular Disease and Metabolic Syndrome

Ageing is in part caused by accumulated DNA damage. Various endogenously arising DNA lesions accumulate with age in the nuclear and mitochondrial genomes of healthy mammals, including humans.

Cell senescence and apoptosis are suspected causes of ageing under conditions where attempted tissue regeneration causes stem-cell exhaustion. Indeed, growing evidence points to human atherosclerosis being characterized by enhanced DNA damage and DDR signalling, leading to senescence of vascular smooth muscle cells and death of other cells to yield atherosclerotic lesions. Modulating ROS production and the DDR therefore represent potential therapeutic opportunities for atherosclerosis.

Metabolic syndrome is a condition characterized by aberrant glucose metabolism, insulin resistance and atherosclerosis. DDR-regulated kinases target multiple substrates involved in glucose metabolism. Although some linkages between the DDR and metabolic syndrome might be indirect, it is possible that the DDR directly modulates certain aspects of energy metabolism and vascular physiology of relevance to metabolic syndrome. (Jackson, Stephen P., and Jiri Bartek. “The DNA-damage Response In Human Biology And Disease.” Nature: 1071-078.)

### Inflammation/Infection

The association between inflammation and oxidative stress is well documented, with studies of inflammatory conditions or infections reporting elevated levels of 8-OH-dG: hepatitis, hepatitis C infection and atopic dermatitis. Mechanistically, chronic inflammation is closely linked to carcinogenesis. Tumor promoters report to recruit inflammatory cells that, with their potential to generate ROS, may provide the appropriate stimuli to lead to promotion. (Cooke, M. S. “Oxidative DNA Damage: Mechanisms, Mutation. And Disease”. The FASEB Journal 2003, 17, 10, 1195-1214.)

### Immune Deficiencies

Genome rearrangements involving DDR factors occur during immune-system development, and these DDR defects cause immune deficiency.

### Alcohol Induced DNA Damage

Chronic alcohol consumption and metabolism result in the generation of several classes of DNA-damaging molecules, including reactive oxygen species (ROS), lipid peroxidation products, and acetaldehyde. Ethanol induction of CYP2E1 increases generation of ROS. DNA strand breaks occurs by means of hydroxyl radical, as it is very sensitive to hydroxyl radical, and which produces over 20 different types of DNA base damage, with diverse biological properties. The hydroxy radical do not diffuse the nucleus but it is generated via H₂O₂ with metal (fenton reaction) as H₂O₂ are highly diffusable in nucleus and produce DNA damage. Apart from that chronic alcoholism causes vitamin and other micro-nutrient deficient state that further aggravates the condition.

Malondialdehyde is the major product generated due to lipid peroxidation on ethanol consumption. Malondialdehyde reacts with guanosine residues in DNA to form malondialdehyde-deoxyguanosine (dG) adduct which is mutagenic and carcinogenic. Another mutagenic lipid peroxidation product due to chronic ethanol consumption is hydroxynonenal (HNE) DNA adducts. Etheno base adducts derived from lipid peroxidation are known carcinogens and mutagens. The mechanism of the mutagenicity of acetaldehyde involves direct attack of acetaldehyde on DNA to give N²-ethyl-deoxyguanosine (N²-Et-dG) even on moderate consumption of ethanol.

Following mentioned patent literature mentions combinations of different compounds which are reported for having DNA protection and/or DNA repair:

EP2218449 discloses use of mannitol in treatment of genome disorders and for DNA damage repair. It is used in treatment of the consequences of ageing and in genome maintenance disorders.

WO2007133076 discloses a combination of mannitol and proline in treatment of DNA damage. The combination is used to treat subjects suffering from genetic defects and ageing.

U.S. Pat. No. 8,911,774 discloses a composition which comprises glycyrrhizin in a concentration range from 0.005% to 0.3% (w/w). This composition provides DNA repair in the nucleus and in the mitochondria.

It is observed that in prior art different components individually are reported, but the extent of DNA protection with these individual components is limited and therefore there is a need for a composition for optimum DNA protection and DNA repair providing maximum health benefits.

### OBJECTS OF THE INVENTION

Primary object of the invention is to provide a composition for DNA protection.

Another object of the invention is to provide a composition for reduction of DNA damage in the body due to endogenous or exogenous factors.

Another object of the invention is to provide a composition for reduction of DNA damage in the body due to alcohol consumption.

A further object of the invention is to provide a composition for repair of damaged DNA in the body.

Another object of the invention is to provide a composition which is effective in reduction of DNA damage and enhances repair of the damaged DNA in the body.

Overall object of the invention is to provide a composition which is effective in reduction of DNA damage due to alcohol consumption and which enhances repair of the damaged DNA in the body and which can be used mixed with the alcohol or alcoholic beverages.

### SUMMARY OF THE INVENTION

Accordingly, invention provides a composition providing synergistic DNA protection comprising:
(a) saponin glycoside in a mass concentration range of 0.01% to 2.0%;
(b) sugar or sugar alcohol in a mass concentration range of 0.01% to 5.0%; and
(c) optionally, flavouring agents and pH adjusting agents;
wherein the composition effectively reduces alcohol-induced DNA damage, repairs damaged DNA and enhances the repair thereof.

The saponin glycoside herein above may comprise Glycyrrhizin (GA) or Glycyrrhizin salt (GA salt), or a combination of Glycyrrhizin (GA) and Glycyrrhizin salt. The Glycyrrhizin (GA) herein comprises 18β-Glycyrrhizin or 18α-Glycyrrhizin, or a combination thereof.

The Glycyrrhizin salt (GA salt) herein comprises 18α-Glycyrrhizin mono ammonium salt, 18β-Glycyrrhizin mono ammonium salt or a combination of 18α-Glycyrrhizin mono ammonium salt and 18β-Glycyrrhizin mono ammonium salt.

In a general embodiment, the Glycyrrhizin (GA) comprises equal parts of 18β-Glycyrrhizin and 18α-Glycyrrhizin and the saponin glycoside is in a mass concentration range of 0.04% to 0.3%.

The sugar in the invention comprises D-Maltodextrin, L-Maltodextrin, D-Maltose, L-Maltose, D-Dextrose, L-Dextrose, D-Glucose, L-Glucose, D-Trehalose, L-Trehalose, D-Sucrose, L-Sucrose, D-Lactose, L-Lactose, Hydrogenated Starch Hydrolysates, D-Fructose or D-Galactose, or a mixture thereof.

The sugar alcohol in the invention comprises D-Glycerol, L-Glycerol, D-Mannitol, L-Mannitol, D-Erythritol, L-Erythritol, D-Xylitol, L-Xylitol, L-Maltitol, D-Maltitol, L-Sorbitol, D-Sorbitol, L-Lactitol, D-Lactitol, L-Isomalt or D-Isomalt, or a mixture thereof.

In a general embodiment, the sugar or sugar alcohol is in a mass concentration range of 0.5% to 2.5%.

In one embodiment, the sugar alcohol is D-Mannitol or L-Mannitol, or a mixture of D-Mannitol and L-Mannitol.

In another embodiment, the sugar alcohol is D-Erythritol or L-Erythritol, or mixture of D-Erythritol and L-Erythritol.

The pH adjusting agent in the invention may be selected from the group consisting of potassium sorbate, monobasic sodium phosphate, dibasic sodium phosphate and tribasic sodium phosphate.

In one embodiment, the pH adjusting agent is potassium sorbate in a mass concentration range of 0.01% to 0.2%.

The flavouring agent in the invention may be selected from the group consisting of extracts of herbs, spices, fruit, and artificial flavour, in mass concentration range of 0.01% to 0.2%.

Accordingly, in one embodiment, the invention provides a composition comprising sugar alcohol in a mass concentration of 0.04% and saponin glycoside in a mass concentration of 0.8%.

In another embodiment, the invention provides a composition comprising sugar alcohol in a mass concentration of 0.04% and saponin glycoside in a mass concentration of 1.2%.

In another embodiment, the invention provides a composition comprising sugar alcohol in a mass concentration of 0.4% and saponin glycoside in a mass concentration of 2.5%.

In another embodiment, the invention provides a composition comprising sugar alcohol in a mass concentration of 0.1% and saponin glycoside in a mass concentration of 0.8%.

In another embodiment, the invention provides a composition comprising sugar alcohol in a mass concentration of 0.1% and saponin glycoside in a mass concentration of 1.2%.

In another embodiment, the invention provides a composition comprising sugar alcohol in a mass concentration of 0.1% and saponin glycoside in a mass concentration of 2.5%.

In another embodiment, the invention provides a composition comprising sugar alcohol in a mass concentration of 0.3% and saponin glycoside in a mass concentration of 0.5%.

In another embodiment, the invention provides a composition comprising sugar alcohol in a mass concentration of 0.3% and saponin glycoside in a mass concentration of 0.8%.

In another embodiment, the invention provides a composition comprising sugar alcohol in a mass concentration of 0.3% and saponin glycoside in a mass concentration of 1.2%.

The composition may be formulated as a beverage additive in a suitable physical form selected from liquid, solid, semi-solid, powder or viscous solid. It may be in the form of tablet, granule or sachet also.

In one embodiment, the beverage additive may be a blend of active ingredients, optionally a pH adjusting agent and a flavouring agent formulated as a solution with a solvent selected from distilled alcohol, deionized water or a combination of deionized water and distilled alcohol. The beverage additive may be mixed with a beverage such as alcoholic beverage and soft-drink beverage.

In one preferred embodiment, the beverage additive is mixed with an alcoholic beverage.

### BRIEF DESCRIPTION OF DRAWINGS

FIG. 1A. represents changes in comet assay at different time intervals after alcohol consumption

FIG. 1B. represents trend line of comet assay at different time intervals after alcohol consumption

FIG. 2A. represents changes in CBMN count at different time intervals after alcohol consumption

FIG. 2B. represents trend line of CBMN assay at different time intervals after alcohol consumption

FIG. 3A. represents changes in serum 8-hydroxy-2-deoxyguanosine assay at different time intervals after alcohol consumption

FIG. 3B. trend line of serum 8OH-dG at different time intervals after alcohol consumption

### DETAILED DESCRIPTION OF THE INVENTION

Inventors of the present invention have found that when a composition of saponin glycoside and sugar or sugar alcohol was formulated with specific concentrations of these components, the composition produced unexpected and surprising results with very high DNA protection both in terms of reduction of DNA damage and repair of the damaged DNA.

Accordingly, the invention relates to a synergistic composition comprising combination of active ingredients to reduce DNA damage, to repair damaged DNA and to enhance repair of damaged DNA. More particularly the invention relates to a synergistic composition comprising combination of active ingredients saponin glycoside and sugar or sugar alcohol compound which is added in a beverage; a beverage additive composition comprising said synergistic combination composition of active ingredients which reduces the DNA damage due to alcohol consumption and due to other reasons; and methods of preparations thereof.

The composition also enhances repair of the DNA which has already been damaged.

The composition and the method of preparation of the composition will now be described, which do not restrict the scope and ambit of the present invention.

The composition may broadly comprise all or some of the active ingredients selected from saponin glycoside, sugar or sugar alcohol compounds.

In one aspect the invention provides a synergistic composition comprising combination of active ingredients
saponin glycoside and
sugar or sugar alcohol compounds
   wherein at appropriate concentration/proportion/amount/quantity of each of the group of ingredients in the combination exhibits synergistic activity and thus synergistically reduces DNA damage due to varied etiology including alcohol consumption and also repairs and/or enhances repair of the DNA which has already been damaged.

Optionally the above composition may further comprise non-active ingredients such as one or more of each of pH adjusting agents and flavouring agents.

The above said synergistic composition of active ingredients with or without non-active ingredients are blended and formulated in suitable physical form. The formulated composition (blend of ingredients) is used as a beverage additive or liquor additive which is added in a beverage/liquor such as alcoholic drink/beverage or any other beverage such as soft drinks, wherein thus obtained beverage composition synergistically reduces DNA damage due of varied etiology including alcohol consumption and also repairs and/or enhances repair of the DNA which has already been damaged.

The above composition may be formulated as a beverage additive or liquor additive in any suitable physical form such that conveniently the composition can be used/added as an additive in a beverage/liquor. Non-limiting suitable physical forms includes liquid, solid, semi-solid, powder or viscous solid. Preferably the composition is formulated as a beverage additive in liquid or solid or powder form.

In one embodiment the beverage additive composition is formulated as a solution in deionized water.

The synergistic composition of the invention having synergistic effect on reducing DNA damage, protection of DNA damage and repairs and/or enhancing repairs thereof can be formulated as a beverage additive which can be later added with a beverage such as alcohol at the time of drinking or can be added to the alcoholic beverage during its preparation itself.

In one embodiment the above said beverage additive is added with alcohol at the time of drinking.

In another embodiment the beverage additive is added with the alcohol during the preparation of the alcoholic beverage.

In any of the above case appropriate % of alcohol that is suitable for drink can be adjusted and used.

Thus in one embodiment of the above aspect the beverage additive composition comprises:
saponin glycoside,
sugar or sugar alcohol compounds,
water (qs),
optionally, a pH adjusting agent,
optionally, a flavouring agent.

In another aspect the invention provides a synergistic beverage composition comprising alcohol and above said beverage/liquor additive, wherein the beverage/liquor additive comprises combination of active ingredients i.e. saponin glycoside and sugar or sugar alcohol compounds, optionally one or more of each of pH adjusting agents and flavouring agents; wherein thus obtained beverage composition synergistically reduces DNA damage of varied etiology including alcohol consumption and also repairs and/or enhances repair of the DNA which has already been damaged.

Thus in one aspect the invention provides a synergistic beverage composition comprising alcohol and active ingredients:
saponin glycoside and
sugar or sugar alcohol compounds.

Optionally the composition may comprise non active ingredients such as one or more of each of pH adjusting agents and flavouring agents.

The beverage composition further comprises quantum sufficient (qs) of water and quantum sufficient (qs) of alcohol.

Thus in one embodiment of the above aspect the synergistic beverage composition comprises:
saponin glycoside,
sugar or sugar alcohol compounds,
alcohol (qs),
water (qs),
optionally, a pH adjusting agent,
optionally, a flavouring agent.

The above beverage composition of the invention may be an alcoholic beverage or any other beverage.

In one embodiment, suitably the beverage composition is an alcoholic beverage composition comprising alcohol as a liquid to be drink.

In another embodiment, the beverage composition may be other than alcoholic beverage also. In such a case when the above beverage composition of the invention is not an alcoholic beverage, the above beverage composition does not comprise alcohol and alcohol is replaced with a suitable potable liquid such as deionized water. In this case also the beverage composition exhibits similar synergistic effects in reducing DNA damage and repairing and/or enhancing repair of damaged DNA. The DNA damage may arise from either alcohol consumption or any other reasons.

### Ingredients, Concentrations and Combinations:

The ingredients of the any of the above compositions
comprise active-ingredients,
may or may not comprise non-active ingredients and
may or may not comprise water or alcohol, or both water and alcohol as required in formulating and preparing the composition.

Active Ingredients: Saponin glycosides and Sugar or Sugar Alcohols.

Non-Active Ingredients: pH adjusting agent and/or flavouring agent.

### Saponin Glycoside:

The saponin glycoside may include Glycyrrhizin (or Glycyrrhizic acid or Glycyrrhizinic acid: abbreviated as GA) is the chief sweet-tasting constituent of Glycyrrhiza glabra (liquorice) root. It has also been given intravenously in Japan as a treatment for hepatitis C and as an emulsifier and gel-forming agent in foodstuff and cosmetics. Glycyrrhizin (GA) is a triterpenoid saponin glycoside. It is available as in racemic or pure form of 2 isomers: 18β-Glycyrrhizin and 18α-Glycyrrhizin. Hepato-protective mechanism of GA is due to its aglycone, glycyrrhetic acid, which inhibits both free radical generation as well as lipid peroxidation. 18α-GA has anti-hepato fibrosis effect—it is frequently used as a hepato-protective agent. The sweetness of GA has a slower onset than sugar, and lingers in the mouth for some time. GA is partly absorbed as an intact drug. (W. Xu-yinga et. al.) Chemico-Biological Interactions 181 (2009) 15-19), (T, Zing et. al., Chinese Journal of Modem Applied Pharmacy 2006, 02, 15-19).

GA induces phase II enzymes involved in the detoxification and excretion of carcinogenic or toxic substances and other antioxidant enzymes responsible for maintaining a balanced state between free radicals/oxidants and the antioxidants within the cellular environment. Oxidative injury in AR mice (allergic rhinitist mice) is reduced by GA, by increasing GSH content and decreased MDA formation in a dose dependent manner. Concomitant decreases were observed in glutathione peroxidase (GPx), catalase (CAT), total antioxidant capacity (TAOC) and SOD activities in AR mice. IFN-α, or type II interferon, is a cytokine that is critical for innate and adaptive immunity against viral and intracellular bacterial infections and for tumour control. (Xiao-Lan Li et al. Int. J. Mol. Sci. 2011, 12, 905 and Xiao-Lan Li et al. Molecules 2012, 17, 716-727).

The saponin glycoside which comprises Glycyrrhizin (GA) or its isomers or its derivatives may be used in the composition in a mass concentration range of 0.01 to 2.0%.

The above said saponin glycoside may comprise Glycyrrhizin (GA) or Glycyrrhizin salt (GA salt), or a combination of Glycyrrhizin (GA) and Glycyrrhizin salt (GA salt).

In an aspect of the present invention the saponin glycoside comprises a glycyrrhizin (GA), such as 18-beta glycyrrhizin (18β-GA), 18-alpha glycyrrhizin (18α-GA), or a combination of 18-beta glycyrrhizin and 18-alpha glycyrrhizin (18β-GA+18α-GA). Alternatively, the saponin glycoside comprises a glycyrrhizin salt (GA salt), such as mono ammonium 18-beta glycyrrhizin salt (mono ammonium 18β-GA salt), mono ammonium 18-alpha glycyrrhizin salt (mono ammonium 18α-GA salt), or a combination of mono ammonium 18-beta and mono ammonium 18-alpha glycyrrhizin. In another aspect, the saponin glycoside comprises a combination of glycyrrhizin (18β-GA or 18α-GA) and a mono ammonium glycyrrhizin salt (salt of 18β-GA or 18α-GA).

When a combination of 18β-Glycyrrhizin and 18α-Glycyrrbizin is used such combination comprises equal parts of 18β-Glycyrrhizin and 18α-Glycyrrhizin i.e. in a ratio of 1:1.

When a combination of 18α-Glycyrrhizin mono ammonium salt and 18β-Glycyrrhizin mono ammonium salt is used such combination comprises equal parts of 18α-Glycyrrhizin mono ammonium salt and 18β-Glycyrrhizin mono ammonium salt.

Under appropriate circumstances, as will be understood by one with ordinary skill in the art, it may be desirable to include a different phytoconstituent extracted from Glycyrrhiza glabra instead of glycyrrhizin, such as liquiritin or another flavonoid, to achieve similar desirable effects.

### Sugar and Sugar Alcohol

A sugar alcohol is a kind of alcohol prepared from sugars. These organic compounds are a class of polyols, also called polyhydric alcohol, polyalcohol, or glycitol. They are white, water-soluble solids that occur naturally and are used widely in the food industry as thickeners and sweeteners. Sugar alcohols such as Mannitol, Erythritol, Sorbitol, Xylitol etc., which are chemically stable can be used as a radical scavengers (hydroxyl radicals). Compounds like Erythritol, Mannitol, Sorbitol, Xylitol etc. up-regulated different types of superoxide dismutase (SOD) like Cu/Zn-, Mn- and EC-SOD isozymes. In particular, the SOD activity of the erythritol-added group increased by 2-5 times. Diabetics have a low SOD activity due to the Maillard reaction, because the Maillard reaction remarkably causes a decrease in the SOD activity (US Patent Application 20100037353 A1). Mannitol containing hyperosmolar solution has been shown to protect ethanol-induced gastric mucosal damage (Gharzouli K, Exp. Toxic. Pathol., 2001; 53: 175). Both rats and humans absorb and metabolize partially the Mannitol ingested in gastro intestinal tract (GIT). However, intestinal microflora convert Mannitol in to more absorbable form. In rat, absorbed mannitol is converted in to hepatic glycogen probably via fructose (J. Nutr. 1985, 115: 890). The mechanism of protecting living cells by Mannitol is not fully understood.

In an aspect of the present invention, the sugar alcohol includes a sugar alcohol, such as D-Mannitol, L-Mannitol, D-sorbitol, L-sorbitol, D-erythritol, D-xylitol, L-xylitol or mixture thereof. Under appropriate circumstances, as will be understood by one with ordinary skill in the art, in order to help achieve the above-mentioned benefits, it is desirable to include a different combination of the aforementioned sugar alcohols

In another aspect of the present invention, the sugar includes, such as D-Xylose, D-Mannose, D-Sucrose, D-Lactose or mixture thereof. Under appropriate circumstances, as will be understood by one with ordinary skill in the art, in order to help achieve the above-mentioned benefits, it is desirable to include a combination of the aforementioned sugars.

The Sugar or Sugar Alcohol may be used in the composition in a mass concentration range of 0.01 to 5.0%. In one embodiment of the invention, any of the above compositions may comprise only one sugar alcohol or sugar. In another embodiment of the invention, any of the above compositions may comprise mixture of two or more sugar alcohol(s) and/or sugar(s). Preferably any of the above compositions may comprise sugar alcohols such as Mannitol (D or L) or Xylitol (D or L) or Erythritol (D or L).

In one preferred embodiment, the sugar alcohol present in any of the above compositions is D-Mannitol. In another preferred embodiment, the sugar alcohol present in any of the above compositions is D-Xylitol. In another preferred embodiment, the sugar alcohol present in any of the above compositions is D-Erythritol.

Further definitions of active ingredients for the groups such as saponin glycoside, sugar and sugar alcohol are as defined below under the heading—Definitions.

Optionally, any of the above said compositions of the invention further comprises other non-active ingredients such as one or more pH adjusting agent(s) and/or one or more flavouring agent(s) each present in an amount ranging from in a mass concentration range of about 0.01 to 0.2%.

### pH Adjusting Agent

The pH adjusting agent is an organic or inorganic base/buffer, which may be selected from potassium sorbate, monobasic sodium phosphate, dibasic sodium phosphate and tribasic sodium phosphate present in a mass concentration range of 0.01 to 0.2%.

In one preferred embodiment the pH adjusting agent which also acts as food preservative is potassium sorbate (KS) present preferably in a mass concentration range of 0.01 to 0.2%. Any suitable amount (%) of pH adjusting agent within the said range (%) can be used based on the requirement of the composition to adjust the pH value in between 4.0 to 10.

### Flavouring Agent

The one or more flavouring agent to impart/improve taste/odour of the composition may be natural flavouring agents such as extracts of herbs, spices or fruit; or artificial or palatable synthetic flavouring agents present in a mass concentration range of about 0.01 to 0.2%. In one embodiment the flavouring agent is selected from vanilla and strawberry. In one embodiment, the composition does not comprise any flavouring agent.

Any suitable amount (%) of flavouring agent within the said range (%) can be used based on the requirement of the composition.

Water and/or Alcohol

The water is deionized water and the alcohol is distilled alcohol. The distilled alcohol may be aqueous distilled alcohol. The quantities of both the water and alcohol are quantity sufficient (qs) and are added to make the composition 100% in total.

When the composition is a beverage additive, the composition may comprise or may not comprise water and/or alcohol. In one embodiment when the beverage additive is formulated as solution, the composition may comprise any one of water and alcohol or comprise both of water and alcohol.

When the composition is a beverage composition including alcoholic beverage composition, the composition may comprise both water and alcohol.

### Synergistic Combinations of Active Ingredients

In any of the above compositions of the invention comprising combination of active ingredients saponin glycoside and sugar or sugar alcohol in appropriate concentration/proportion/amount/quantity of each of the active-ingredients exhibits synergistic biological activity.

The composition comprises combination of two active-ingredients—one from saponin glycoside and one from sugar or sugar alcohol. The saponin glycoside and sugar or sugar alcohols are as defined above.

Appropriate concentration/proportion/percentage/quantity/amount is the amount of each active agent that must be included in a composition to effectively result in a net DNA protective effect or DNA repair effect or enhancing DNA repair synergistically.

Effective appropriate amount of Saponin Glycoside such as Glycyrrhizin or its derivatives or its salt is in a mass concentration range anywhere in between 0.01% to 2.0%.
Without any limitation, the effective amount of Saponin Glycoside including GA or GA Salt or combination of GA and GA Salt may be in a range (%) one selected from ranges 0.01 to 2.0% or 0.01 to 1.5% or 0.01 to 1.0% or 0.01 to 0.75% or 0.01 to 0.5% or 0.01 to 0.3% or 0.04 to 0.3%.
In one preferred embodiment, the % of GA or GA Salt or combination of GA and GA Salt may be 0.01% or 0.02% or 0.03% or 0.04% or 0.05% or 0.06% or 0.07% or 0.08% or 0.09% or 0.1% or 0.11% or 0.12% or 0.13% or 1.14% or 0.15% or 0.16% or 0.17% 0.18% or 0.19% or 0.2% or 0.21% or 0.22% or 0.23% or 0.24% or 0.25% or 0.26% or 0.27% or 0.28% or 0.29% or 0.3%.
Effective appropriate amount of Sugar alcohol such as Mannitol or Xylitol or Erythritol or sugar is in a mass concentration range anywhere in between 0.01% to 5.0%.
Without any limitation, the effective amount of sugar alcohol or sugar may be a range (%) one selected from 0.01 to 5.0% or 0.1 to 3.0% or 0.1 to 2.5% or 0.5 to 2.5%.
In one preferred embodiment, the % of sugar alcohol including Mannitol or Xylitol or Erythritol or % of sugar or combination of sugar alcohol and sugar may be 0.1% or 0.2% or 0.3% or 0.4% or 0.5% or 0.6% or 0.7% or 0.8% or 0.9% or 1.0% or 1.1% or 1.2% or 1.3% or 1.4% or 1.5% or 1.6% or 1.7% or 1.8% or 1.9% or 2.0% or 2.1% or 2.2% or 2.3% or 2.4% or 2.5% or 2.6% or 2.7% or 2.8% or 2.9% or 3.0%.

In one embodiment the invention provides a beverage additive composition comprising:
glycyrrhizin (GA) or its derivatives or its pharmaceutically salt in a mass concentration range of 0.01 to 2.0%;
a sugar alcohol or sugar in a mass concentration range of 0.01 to 5.0%;
optionally, pH adjusting agent(s) in a mass concentration range of 0.01 to 0.2%; and
optionally, flavouring agent(s) in a mass concentration range of 0.01 to 0.2%.
When the above beverage additive composition is formulated as a solution, it may comprise or may not comprise any of water and alcohol or both of water and alcohol.

In one embodiment the invention provides a beverage composition comprising:
glycyrrhizin (GA) or its derivatives or its pharmaceutically salt in a mass concentration range of 0.01 to 2.0%,
a sugar alcohol or sugar in a mass concentration range of 0.01 to 5.0%,
a quantum sufficit (qs) of deionized water,
a quantum sufficit (qs) of distilled alcohol,
optionally, pH adjusting agent(s) in a mass concentration range of 0.01 to 0.2%, and
optionally, flavouring agent(s) in a mass concentration range of 0.01 to 0.2%.

In any of the above presented embodiments, the concentrations/amounts of active ingredients are in effective amount within the above mentioned ranges or mentioned preferred % of active ingredients.

Further the appropriate amounts of Saponin Glycoside and Sugar/Sugar Alcohol in the combination may be the amounts as depicted in Table-1

In one embodiment, any of the above compositions comprises one combination of saponin glycoside and sugar alcohol selected from: 18β-GA+Mannitol, 18β-GA+Xylitol, 18β-GA+Erythritol, 18α-GA+Mannitol, 18α-GA+Xylitol, 18α-GA+Erythritol, 18β-GA:18α-GA (1:1)+Mannitol, 18β-GA:18α-GA (1:1)+Xylitol and 18β-GA: 18α-GA (1:1)+Erythritol.

In one embodiment, any of the above compositions comprises below appropriate effective amounts of combination of active ingredients:
(a) 18β-GA or 18α-GA (0.04-0.3%)+D-Mannitol or D-Erythritol (0.5-2.5%) or
(b) 18β-GA:18α-GA (1:1) (0.04-0.3%)+D-Mannitol or D-Erythritol (0.5-2.5%)

The above mentioned combinations of two main active ingredients are non-limiting and are illustrative. Without limiting with above, the composition or beverage additive composition and/or the beverage composition of the invention may comprise any appropriate and effective amounts of said two groups of active ingredients, which are capable to show greater protection and show greater synergism. Further the above combination of two groups of active ingredients in the beverage additive composition and/or the beverage composition optionally comprises appropriate amounts of pH adjusting agent(s) and flavouring agents(s).

In another embodiment of the invention any of the above synergistic composition, synergistic beverage additive composition and synergistic beverage composition comprises any one of below combination of active ingredients along with or without water and/or alcohol, optionally along with other non-active ingredients such as one or more pH adjusting agents and/or flavouring agents:
a. 18β-GA or 18α-GA or 18β-GA:18α-GA (1:1) in a mass concentration of 0.04% and D-Mannitol or L-Mannitol or D-Erythritol or L-Erythritol in a mass concentration of 0.8%.
b. 18β-GA or 18α-GA or 18β-GA: 18α-GA (1:1) in a mass concentration of 0.04% and D-Mannitol or L-Mannitol or D-Erythritol or L-Erythritol in a mass concentration of 1.2%.
c. 18β-GA or 18α-GA or 18β-GA: 18α-GA (1:1) in a mass concentration of 0.04% and D-Mannitol or L-Mannitol or D-Erythritol or L-Erythritol in a mass concentration of 2.5%.
d. 18β-GA or 18α-GA or 18β-GA: 18α-GA (1:1) in a mass concentration of 0.1% and D-Mannitol or L-Mannitol or D-Erythritol or L-Erythritol in a mass concentration of 0.8%.
e. 18β-GA or 18α-GA or 18β-GA:18α-GA (1:1) in a mass concentration of 0.1% and D-Mannitol or L-Mannitol or D-Erythritol or L-Erythritol in a mass concentration of 1.2%.
f. 18β-GA or 18α-GA or 18β-GA:18α-GA (1:1) in a mass concentration of 0.1% and D-Mannitol or L-Mannitol or D-Erythritol or L-Erythritol in a mass concentration of 2.5%.
g. 18β-GA or 18α-GA or 18β-GA:18α-GA (1:1) in a mass concentration of 0.3% and D-Mannitol or L-Mannitol or D-Erythritol or L-Erythritol in a mass concentration of 0.5%.
h. 18β-GA or 18α-GA or 18β-GA:18α-GA (1:1) in a mass concentration of 0.3% and D-Mannitol or L-Mannitol or D-Erythritol or L-Erythritol in a mass concentration of 0.8%.
i. 18β-GA or 18α-GA or 18β-GA:18α-GA (1:1) in a mass concentration of 0.3% and D-Mannitol or L-Mannitol or D-Erythritol or L-Erythritol in a mass concentration of 1.2%.

To measure the % protection and % synergistic effect of the above said compositions comprising combinations of saponin glycoside and sugar alcohol, different combinations with alterations in amount/quantity of each ingredients are tested and evaluated for its effects on DNA damage. The results show excellent synergistic effect of combination of two active ingredients from two groups viz. Saponin glycoside and sugar alcohol or sugar at effective concentrations/amounts as compared to effects of any single active ingredient (Results in Table-1).

### Experimental Studies

Various combination of the formulation comprising of two compounds from group of category such as a sugar alcohol or sugar, and Saponin glycoside in calculated proportions in the alcohol exhibits a synergistic effect which alleviates oxidative stress, DNA damage parameter, which leads to preventing alcohol-induced damage or impairment of organs, which could be temporary or permanent. It has unexpectedly been found that the combination of these components, in synergistically effective quantities, demonstrates statistically significant improvement in selected DNA damage recovery, which would be expected as an additive effect of the cumulative protecting effect of each individual component (active ingredient). In addition, a composition of a sugar alcohol or sugar, and Saponin glycoside in alcohol has also been found to reduce stress.

Further, an unexpected synergy is observed when an alcoholic beverage composition including these class of compounds are consumed over the alcoholic beverage composition including these class of compounds incorporated separately.

The sequential consumption of the synergistic composition simultaneously with alcohol improves the physiological and psychological parameters, bringing them closer to normal values. The toxicity of the alcoholic beverage is significantly reduced.

### Evaluation of DNA Damage

A design of experiments (“DOE”) methodology, a standard methodology of experimentation used to obtain the maximum information from the minimum number of experiments, was employed to investigate the performance of the composition for modulating immunology parameters, alleviating oxidative stress, DNA damage.

To evaluate DNA damage, one or more of the following methodology was used:
1. Comet assay studies from peripheral lymphocytes.
2. 8-OHdG,

1. Comet Assay:

The Single Cell Gel Electrophoresis assay (SCGE, also known as comet assay) is a straightforward and sensitive technique for the detection of DNA damage at the level of the individual eukaryotic cell. It is being used as a standard technique for evaluation of DNA damage/repair, biomonitoring and genotoxicity testing. It involves the encapsulation of cells in a low-melting-point agarose suspension, lysis of the cells in neutral or alkaline (pH>13) conditions, and electrophoresis of the suspended lysed cells. The term “comet” refers to the pattern of DNA migration through the electrophoresis gel, which often resembles a comet (Cheng-Hung Chuang, Miao-Lin Hu. Use of whole blood directly for single-cell gel electrophoresis (comet) assay in vivo and white blood cells for in vitro assay. Mutation Research; 564 (2004) 75-82.).

2. 8-OHdG:

In nuclear and mitochondrial DNA, 8-hydroxy-2′-deoxyguanosine (8-OHdG) is one of the predominant forms of free radical-induced oxidative lesions, and is used as a biomarker for oxidative stress. Rat 8-OHdG ELISA kit from Elabscience is used for the assay. (Valavanidis A et al (2009). 8-hydroxy-2′-deoxyguanosine (8-OHdG): A critical biomarker of oxidative stress and carcinogenesis. J Environ Sci Health C Environ Carcinog Ecotoxicol Rev. 27(2):1120-139).

Several beverage compositions were prepared and evaluated for establishing alleviation of DNA damage.

### Methods of Preparation:

a) Beverage Additive

An embodiment of the method of preparation includes the following steps.

The ingredients which comprise sugar alcohol and saponin glycoside, were mixed in a reactor comprising water or alcohol or both. The components were added portion wise in small quantities. A food preservative like potassium sorbate was added and the pH was adjusted to be in the range from 4.0 to 10.0. A flavouring agent was optionally added. The dissolved formulation was filtered using various filtering techniques and then the clear solution was bottled (as beverage additive).

b) Alcoholic Beverage
The ingredients which comprise sugar alcohol and saponin glycoside were mixed in a reactor containing alcohol and water. The components were added portion wise in small quantities. A food preservative like potassium sorbate was added and the pH was adjusted to be in the range from 4.0 to 10.0. A flavouring agent was optionally added. The dissolved formulation was filtered using various filtering techniques and then the clear solution was bottled (alcoholic beverage/drink).

There could be other variations of preparing the composition and only two embodiments of preparations are disclosed here.

(I) Animal Studies

### Materials and Methods

Animal Studies were Approved by Animal Ethics Committee (Reference No-PHARMA/327/IAEC; Dated 18 Mar. 2015).

### Reagents

Distilled ethanol was obtained from Bengal Chemicals, West Bengal, India. 8-hydroxy-2-deoxyguanosine (8-OHdG) ELISA kit was procured from Elabscience Biotechnology Co. Ltd, WuHan, P.R.C. All the chemicals used in the present study were of analytical grade and obtained from the following companies: Sigma (St. Louis, Mo., USA), Merck (Mumbai, India), S D fine chemicals (Mumbai, India) and Qualigen (Mumbai, India).

(1) DNA Damage Study Models in Rats

(a) Animals

Male Wistar albino rats weighing 150-200 g were procured from local registered traders (CPCSEA Regd No. 1443/po/6/4/CPCSEA), Kolkata, India and were acclimatized for 7 days at standard housing condition (26° C.±2° C., 60-70% RH with 12±1 hours light and dark cycle). Animals were fed with commercially available diet (Lipton India Pvt. Ltd, India) and water ad-libitum during the experiment period.

### Experimental Design

### Animal Study Model 1:

Saline/Alcohol (4.0 gm/kg/day, p.o.)/Formulation with alcohol (4.0 gm/kg/day, p.o.) were administered (fed orally by orogastric cannula) for one day. Blood samples were collected before administration of the Saline/Alcohol/Formulation with alcohol and designated as ‘0’ hour, then 2 and 4 hours after the dose. For comet assay one part of the blood was collected in heparinised tube and the other part was allowed to clot to get serum for 8-OHdG assay. Single cell gel electrophoresis (SCGE, the Comet assay) was carried out using whole blood (Cheng-Hung Chuang, Miao-Lin Hu. Use of whole blood directly for single-cell gel electrophoresis (comet) assay in vivo and white blood cells for in vitro assay. Mutation Research; 564 (2004) 75-82.). In short, 15 μl of whole blood was mixed with low melting agarose and fixed on a microscope slide. Those are then lysed by buffer enriched with detergent and high salt. Concomitant electrophoresis at alkaline pH results in unwinding of DNA followed by structures close to comets, when observed by fluorescence microscopy. The intensity of the comet tail relative to the head presents the number of DNA breaks. The probable basis for this is that loops containing a break lose their super coiling and become free to extend toward the anode. This is followed by visual analysis with staining of DNA and calculating fluorescence to determine the extent of DNA damage (Collins, A. R. (2004). “The comet assay for DNA damage and repair: principles, applications, and limitations”. Mol. Biotechnol. 26 (3): 249-261). 8-hydroxy-2-deoxyguanosine was estimated (8-OHdG) by ELISA kit using competitive-ELISA as the method. The microtiter plate is pre-coated with 8-OHdG. 8-OHdG in the sample or standard competes with a fixed amount of 8-OHdG on the solid phase supporter for sites on the Biotinylated Detection Ab specific to 8-OHdG. Excess conjugate and unbound sample or standard were washed from the plate, and Avidin conjugated to Horseradish Peroxidase (HRP) is added to each microplate well. After incubation a TMB substrate solution is added to each well. The enzyme-substrate reaction is terminated by the addition of a sulphuric acid solution and the color change was measured by plate reader at a wavelength of 450 nm±2 nm. The concentration of 8-OHdG in the samples was then determined by comparing the OD of the samples to the standard curve (Szymańska-Chabowska A et al, Evaluation of DNA Damage in People Occupationally Exposed to Arsenic and Some Heavy Metals. Polish J. of Environ. Stud. Vol. 18, No. 6 (2009), 1131-1139).

### % Protection Calculated in Above Experiments:

### % Protection=1−[(T−NS)/(AL−NS)]×100

T=Mean value of drug treated, NS=Mean value of normal control, AL=Mean value of alcohol alone.

### EXAMPLES

### Example 1: (DNA Protection Study)

a) Model for Biological Testing:

Male Wistar albino rats weighing 150-200 g were procured and randomly divided into groups consisting of four (4) animals in each group. DNA damage was induced by alcohol in rats by oral administration of 30% alcohol (4 gm/kg/p.o.) once (single dose) and this group served as the negative control and treated groups received different formulations.

b) Preparation of Drug Solution:

All drug solutions were prepared in 40% aqueous alcohol, adjusting the pH in the range of 5.0-10.0 for evaluation of alcohol induced DNA damage and protection against it. This solution was further diluted with distilled water to obtain 30% aqueous alcoholic solution and administered orally by gavage to different rats group of step (1a).

c) Evaluation of DNA Protecting Activity:

Before administration of alcohol and different drug solutions (mentioned in 1b), blood was collected from tail vein of the rats and designated as ‘0’ hr followed by blood collection at 2 hr and 4 hr after administration of the drug solutions. The whole blood (uncoagulated) and the serum were used for comet assay and 8-hydroxyguanosine (8-OHDG) estimation, respectively.

### Example 2: (General Preparations/Formulations)

### Single Active Ingredient: Sugar Alcohol/Saponin Glycoside

(a) Sugar Alcohol (D-Mannitol or D-Erythritol or other) 0.5 g to 2.5 g was dissolved in aqueous alcohol (100 ml) to provide a corresponding 0.5% to 2.5% solution. This solution was administered in several portions to one of the rats group of Example (1a). The administration was carried out over as per example 1(a). Evaluation of DNA damage/protection activities were carried out as per Example (1c).
(b) Saponin Glycoside (18α-GA or 18β-GA or 18α-GA+18β-GA (1:1) or other) 0.04 g to 0.3 g was dissolved in aqueous alcohol (100 ml) to provide a corresponding 0.04% to 0.3% solution. This solution was administered in several portions to one of the rats group of Example (1a). The administration was carried out over as per example 1(a) Evaluation of DNA damage/protection activities were carried out as per Example (1c).

### Two Active Ingredients: Sugar Alcohol and Saponin Glycoside

(c) Sugar Alcohol (D-Mannitol or D-Erythritol or other) 0.5 g to 2.5 g and Saponin Glycoside (18α-GA or 18β-GA or 18α-GA+18β-GA (1:1) or other) 0.04 g to 0.3 g were dissolved in aqueous alcohol (100 ml) to provide a corresponding 0.54% to 2.8% solution. This solution was administered in several portions to one of the rats group of Example (1a). The administration was carried out over as per example 1(a) Evaluation of DNA damage/protection activities were carried out as per Example (1c).

Preparations/formulations comprising a single active ingredient and two active ingredients as mentioned in the Table-1 and also additionally optionally comprising a pH adjusting agent and/or flavouring agent can be prepared and biological evaluation can carried out by the way as described in above examples 1-2 and also as per method of preparation as described above. Further non-limiting specific examples are as given below. The examples below are illustrative and in similar way other combinations and/or beverage additive or beverage comprising combinations of active ingredients can be prepared by taking suitable amounts of active ingredients and optionally suitable amounts of pH adjusting agents and/or flavouring agents.

### Example-3

D-Mannitol or D-Erythritol (0.8 g) was dissolved in aqueous alcohol (100 ml) to provide a corresponding 0.8% solution. This solution was administered in several portions to one of the rats group of Example (1a). The administration was carried out over as per example 1(a). Evaluation of DNA damage/protection activities were carried out as per Example (1c).

### Example-4

D-Mannitol or D-Erythritol (1.2 g) was dissolved in aqueous alcohol (100 ml) to provide a corresponding 1.2% solution. Administration of drug to animals and biological evaluation were carried out as performed Example 3.

### Example-5

D-Mannitol or D-Erythritol (2.5 g) was dissolved in aqueous alcohol (100 ml) to provide a corresponding 2.5% solution. Administration of drug to animals and biological evaluation were carried out as performed in Example 3.

### Example-6

18α-GA or 18β-GA or 18α-GA: 18β-GA (1:1) (0.1 g) was dissolved in aqueous alcohol (100 ml) to provide a corresponding 0.1% solution. Administration of drug to animals and biological evaluation were carried out as performed in Example 3.

### Example-7

18α-GA or 18β-GA or 18α-GA: 18β-GA (1:1) (0.04 g) was dissolved in aqueous alcohol (100 ml) to provide a corresponding 0.04% solution. Administration of drug to animals and biological evaluation were carried out as performed in Example 3.

### Example-8

18α-GA or 18β-GA or 18α-GA:18β-GA (1:1) (0.3 g) was dissolved in aqueous alcohol (100 ml) to provide a corresponding 0.3% solution. Administration of drug to animals and biological evaluation were carried out as performed in Example 3.

### Example-9

18α-GA or 18β-GA or 18α-GA:18β-GA(1:1) (0.04 g) and D-Mannitol (0.8 g) was dissolved in aqueous alcohol (100 ml) to provide a corresponding 0.84% solution. Administration of drug to animals and biological evaluation were carried out as performed in Example 3.

### Example-10

18α-GA or 18β-GA or 18α-GA:18β-GA(1:1) (0.04 g) and D-Mannitol (1.2 g) was dissolved in aqueous alcohol (100 ml) to provide a corresponding 1.24% solution. Administration of drug to animals and biological evaluation were carried out as performed in Example 3.

### Example-11

18α-GA or 18β-GA or 18α-GA: 18β-GA(1:1) (0.04 g) and D-Mannitol (2.5 g) was dissolved in aqueous alcohol (100 ml) to provide a corresponding 2.54% solution. Administration of drug to animals and biological evaluation were carried out as performed in Example 3.

### Example-12

18α-GA or 18β-GA or 18α-GA:18β-GA(1:1) (0.1 g) and D-Mannitol (0.8 g) was dissolved in aqueous alcohol (100 ml) to provide a corresponding 0.9% solution. Administration of drug to animals and biological evaluation were carried out as performed in Example 3.

### Example-13

18α-GA or 18β-GA or 18α-GA:18β-GA(1:1) (0.1 g) and D-Mannitol (1.2 g) was dissolved in aqueous alcohol (100 ml) to provide a corresponding 1.3% solution. Administration of drug to animals and biological evaluation were carried out as performed in Example 3.

### Example-14

18α-GA or 18β-GA or 18α-GA:18β-GA(1:1) (0.3 g) and D-Mannitol (1.2 g) was dissolved in aqueous alcohol (100 ml) to provide a corresponding 1.5% solution. Administration of drug to animals and biological evaluation were carried out as performed in Example 3.

### Example-15

18α-GA or 18β-GA or 18α-GA:18β-GA(1:1) (0.3 g) and D-Erythritol (0.8 g) was dissolved in aqueous alcohol (100 ml) to provide a corresponding 1.1% solution. Administration of drug to animals and biological evaluation were carried out as performed in Example 3.

### Example-16

18α-GA (0.05 g) and 18β-GA (0.05 g) and D-Mannitol (1.2 g) was dissolved in aqueous alcohol (100 ml) to provide a corresponding 1.3% solution. Administration of drug to animals and biological evaluation were carried out as performed in Example 3.

### Example-17

18α-GA (0.15 g) and 18β-GA (0.15 g) and D-Mannitol (0.5 g) was dissolved in aqueous alcohol (100 ml) to provide a corresponding 0.8% solution. Administration of drug to animals and biological evaluation were carried out as performed in Example 3.

In the similar way as exemplified above other preparations comprising either a single active ingredient or combination of two active ingredients, selected from saponin glycoside and sugar or sugar alcohol; can be prepared by taking appropriate required concentration/amount of each ingredients.

The comparative tests results of any single active ingredient and combination of two active ingredients (one from each group) comprising different combinations at different amounts show increased protection (%) and synergism (%) of various test parameters in case of two active ingredients combination each at appropriate amount are depicted in below Table-1.

TABLE 1
STUDY: DNA PROTECTIVE ACTIVITY

| | | COMET | COMET | 8OHdG | 8OHdG |
|---|---|---|---|---|---|
| Sl. No. | Groups | Protection | Synergism | Protection | Synergism |
| | Normal | 100 | | 100 | |
| | Alcohol | 0 | | 0 | |
| | 18 β-GA % or Man % or | | | | |
| | 18 β-GA % + Man % | | | | |
| 1 | 0.04 18β-GA | 0 | | 0 | |
| 2 | 0.1 18β-GA | 0.97 | | 3.55 | |
| 3 | 0.3 18β-GA | 6.07 | | 11.24 | |
| 4 | 0.5 Man | 5.34 | | 7.69 | |
| 5 | 0.8 Man | 9.22 | | 14.20 | |
| 6 | 1.2 Man | 13.59 | | 24.85 | |
| 7 | 2.5 Man | 33.98 | | 40.24 | |
| 8 | 0.04 18β-GA + 0.8Man | 12.38 | 34.21 | 18.34 | 29.17 |
| 9 | 0.04 18β-GA + 1.2Man | 18.69 | 37.50 | 28.40 | 14.29 |
| 10 | 0.04 18β-GA + 2.5Man | 30.58 | −10.00 | 44.97 | 11.76 |
| 11 | 0.1 18β-GA + 0.8Man | 12.62 | 23.82 | 21.30 | 20.00 |
| 12 | 0.1 18β-GA + 1.2Man | 19.90 | 36.67 | 34.91 | 22.91 |
| 13 | 0.1 18β-GA + 2.5Man | 38.11 | 9.03 | 49.11 | 12.16 |
| 14 | 0.3 18β-GA + 0.5Man | 9.22 | −19.16 | 23.67 | 25.03 |
| 15 | 0.3 18β-GA + 1.2Man | 28.16 | 43.21 | 44.38 | 22.96 |
| | Ery % or 18 β-GA % + Ery % | | | | |
| 16 | 0.8 Ery | 8.25 | | 15.98 | |
| 17 | 1.2 Ery | 18.69 | | 23.08 | |
| 18 | 2.5 Ery | 34.71 | | 37.28 | |
| 19 | 0.04 18β-GA + 1.2 Ery | 18.45 | 1.3 | 22.49 | −2.58 |
| 20 | 0.3 18β-GA + 0.8 Ery | 19.42 | 35.6 | 33.95 | 17.40 |
| 21 | 0.1 18β-GA + 2.5 Ery | 35.92 | 0.7 | 51.48 | 26.08 |
| | 18α-GA % or | | | | |
| | 18α-GA % + Man % | | | | |
| 22 | 0.1 18α-GA + 1.2 Man | 18.95 | 33.6 | 32.78 | 12.84 |
| 23 | 0.1 18α-GA | 0.59 | | 4.20 | |
| 24 | 0.1 18α-GA + 2.5 Man | 34.41 | −0.5 | 47.59 | 7.09 |
| 25 | 0.3 18α-GA + 0.5 Man | 10.39 | −6.6 | 22.85 | 25.90 |
| 26 | 0.3 18α-GA | 5.78 | | 10.46 | |
| | 18α:18βGA (1:1) % + Man % | | | | |
| 27 | 0.1 18α:18βGA (1:1) + 1.2 Man | 19.25 | 33.8 | 35.23 | 16.62 |
| 28 | 0.1 18α:18βGA (1:1) | 1.02 | | 5.36 | |
| 29 | 0.3 18α:18βGA (1:1) + 0.5 Man | 10.59 | −14.9 | 25.14 | 23.36 |
| 30 | 0.3 18α:18βGA (1:1) | 7.31 | | 12.69 | |

DNA protecting score was calculated against 4 hr data (n = 4 in each group)
GA: Glycyrrhizin, Man: Mannitol, Ery: Erythritol, Values are in %

### DISCUSSION (Animal Study)

Several combination of 18β and 18 α GA with Mannitol and erythritol and each component alone have been tested for their DNA damage protecting activity in comet assay and 8OHdG assay. The result of comet assay showed that maximum protection with 0.1% 18 β GA+2.5% Man followed by 2.5% Man alone. However, synergism (43.21%) in comet protection was highest with 0.3% 18β-GA+1.2% Man. Combination of 0.04%18β-GA and 0.8% Man exhibited highest synergism (29.17%) in 8 OHdG assay. The results indicate a slightly higher concentration range of GA and moderate concentration range of Mannitol may display some synergistic action in DNA damage protection in comet assay and 8-OHdG assay.

(II) Human Trial

In order to evaluate the efficacy of the composition of the present invention, i.e. composition comprising saponin glycoside and sugar or sugar alcohol, a double blind, randomized cross over trial (Phase II, December, 2015 to August, 2016) was performed wherein the extent of DNA damage from blood after oral consumption of two different alcohol formulations were evaluated.

| Formulations | Components |
|---|---|
| Formulation 1 | Beverage Additive (Glycyrrhizin + Mannitol + Potassium |
| | sorbate) + Base Alcohol |
| Formulation 2 | Base alcohol (No Beverage Additive) |

After preliminary screening of 60 subjects (visit 1) having history of occasional alcohol drinking. 53 subjects (visit 2) were randomly divided in two groups—
a) Beverage Additive Group: Served with Formulation 1
b) Base Alcohol Group: Served with Formulation 2

After seven days washout period (after visit 2) cross over study was performed (visit 3) wherein a) Beverage Additive group was served with Formulation 2 (base alcohol) and b) Base Alcohol group was served with Formulation 1. Total 50 subjects completed the crossover study and 3 subjects dropped out from the study by due to lost to follow-up, unrelated with any adverse event, but due to loss of interest (unwilling to allow multiple blood withdrawal). The subjects were monitored for safety during the study period and two-four hours after the end of the study. Standard study protocols were followed for inclusion and exclusion criteria.

### Biochemical Parameters

Following biochemical markers were evaluated to screen the extent of DNA damage:

Markers of DNA Damage:
a. Single cell gel electrophoresis (SCGE, the Comet assay): Peripheral lymphocytes were fixed in agarose on a microscope slide are lysed with detergent and high salt. Electrophoresis at alkaline pH results in structures close to comets, when observed by fluorescence microscopy. The intensity of the comet tail relative to the head presents the number of DNA breaks. The probable basis for this is that loops containing a break lose their super coiling and become free to extend toward the anode. This is followed by visual analysis with staining of DNA and calculating fluorescence to determine the extent of DNA damage. This was performed using imaging software CaspLab version 1.2.3.beta 1 (Collin A R, 2004).
b. Cytokinesis-Block Micronucleus (CBMN) Assay (selected points): CBMN assay is based on assessment of micronuclei in nucleated cells that have completed only one nuclear division. Human peripheral lymphocytes were cultivated for 72 hours. Blood lymphocytes are stimulated with phytohemagglutinin and their cytokinesis is blocked by cytochalasin B. After cultivation, cultures are hypotonically treated, dropped onto glass slides and stained with Giemsa. Slides are evaluated by microscope. A total of 100 binucleated cells are evaluated for the frequency of appearance of micronucleus (Pejchal J et al, 2011).
c. 8-hydroxy-2-deoxyguanosine was estimated (8-OHdG) by ELISA kit using competitive-ELISA as the method. The microtiter plate has been pre-coated with 8-OHdG. 8-OHdG in the sample or standard competes with a fixed amount of 8-OHdG on the solid phase supporter for sites on the Biotinylated Detection Ab specific to 8-OHdG. Excess conjugate and unbound sample or standard were washed from the plate, and Avidin conjugated to Horseradish Peroxidase (HRP) is added to each microplate well. After incubation a TMB substrate solution is added to each well. The enzyme-substrate reaction is terminated by the addition of a sulphuric acid solution and the color change was measured plate reader at a wavelength of 450 nm±2 nm. The concentration of 8-OHdG in the samples was then determined by comparing the OD of the samples to the standard curve.

### Statistical Analysis

Paired ‘student's t-test’ was carried out between the values of each time points of two groups (Base alcohol and Beverage Additive (BA)) and p<0.05 was considered statistically significant.

### DNA Damage Study

### Single Cell Comet Assay of Peripheral Lymphocytes:

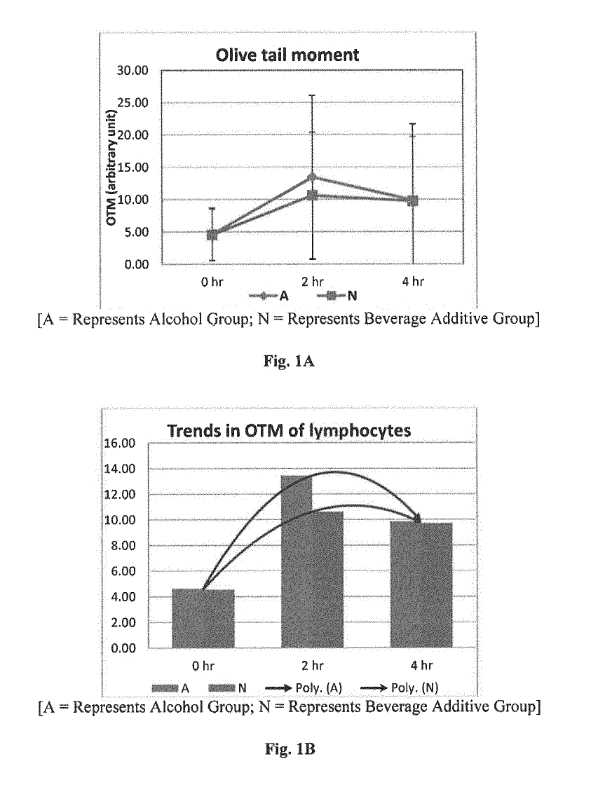

Changes were observed at different time intervals of the oral consumption of alcohol with or without Beverage Additive (BA) groups and were compared in table-2 and FIG. 1A and FIG. 1B.

TABLE 2
Changes in DNA comet score (olive tail
moment) after alcohol consumption

| | Alcohol¹ | BA¹ | | | p value | |
|---|---|---|---|---|---|---|
| Time | (mean | (mean | | | (paired | |
| points | value) | value) | SD-Al | SD-BA | t-test) | Sig.² |
| 0 hr | 4.63 | 4.56 | 4.10 | 3.96 | 0.679 | NS |
| 2 hr | 13.44 | 10.62 | 12.71 | 9.77 | 0.004 | S |
| 4 hr | 9.85 | 9.73 | 11.83 | 9.94 | 0.922 | NS |

Note:
¹n = 50 subjects in each arm completed the study.
Note:
²Significance testing was performed using Paired Sample t-Test, p value < 0.05 is considered as significant

### Cytokinesis-Block Micronucleus (CBMN) Assay of Peripheral Lymphocytes:

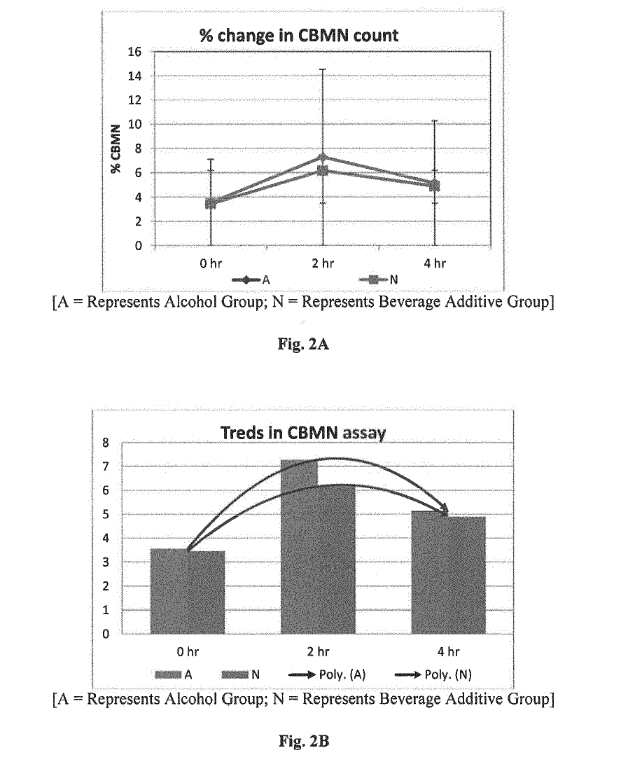

Changes were observed at different time intervals of the oral consumption of alcohol with or without Beverage Additive groups and were compared in table-3 and FIG. 2A and FIG. 2B.

TABLE 3
Changes in micronucleus score (%) after alcohol consumption

| | Alcohol¹ | BA¹ | | | p value | |
|---|---|---|---|---|---|---|
| Time | (mean | (mean | | | (paired | |
| points | value) | value) | SD-Al | SD-BA | t-test) | Sig.² |
| 0 hr | 3.56 | 3.46 | 1.93 | 1.85 | 0.403 | NS |
| 2 hr | 7.28 | 6.16 | 4.08 | 3.43 | 0.013 | S |
| 4 hr | 5.14 | 4.9 | 2.81 | 2.57 | 0.497 | NS |

Note:
¹n = 50 subjects in each arm completed the study.
Note:
²Significance testing was performed using Paired Sample t-Test, p value < 0.05 is considered as significant

### Serum 8-Hydroxy-2-Deoxyguanosine (8OH-dG) (Ng/Ml) Assay:

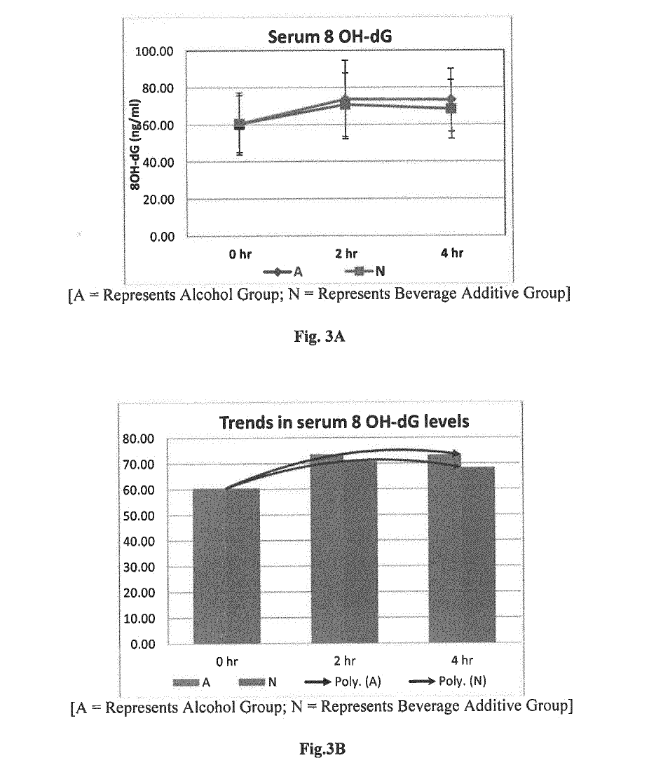

Changes were observed at different time intervals of the oral consumption of alcohol with or without Beverage Additive groups and were compared in table-4 and FIG. 3A and FIG. 3B.

TABLE 4
Changes in serum 8-hydroxy-2-deoxyguanosine
(ng/ml) after alcohol consumption

| | Alcohol¹ | BA¹ | | | p value | |
|---|---|---|---|---|---|---|
| Time | (mean | (mean | | | (paired | |
| points | value) | value) | SD-Al | SD-BA | t-test) | Sig.² |
| 0 hr | 60.52 | 60.44 | 16.71 | 15.26 | 0.913 | NS |
| 2 hr | 73.61 | 70.75 | 21.19 | 17.13 | 0.071 | NS |
| 4 hr | 73.23 | 68.34 | 16.96 | 15.83 | 0.001 | S |

Note:
¹n = 50 subjects in each arm completed the study.
Note:
²Significance testing was performed using Paired Sample t-Test, p value < 0.05 is considered as significant

### DISCUSSION (Human Trial)

DNA protecting effects of Beverage Additive were evaluated by estimating single cell comet assay and cytokinesis-block micronucleus (CBMN) assay from peripheral lymphocytes and serum 8-hydroxy-2-deoxyguanosine (8 OH-dG) assay. A transient rise in olive tail moment (comet score) was observed in both the groups at two hour time point compared to the base line value. Alcohol containing Beverage Additive showed significant slower increase (113%) compared to that of only alcohol group (190%). The similar observation was noted in micronucleus scores. However, at 4 hour time points both the groups showed similar values. The findings indicated that Beverage Additive could able to prevent, at least to a certain extent, the initial damaging effects of alcohol. 8 OH-dG level of alcohol group was increased by 21-22% throughout the post alcohol consumption period whereas the increase of 8 OH-dG levels in Beverage Additive group significantly came down to 13% after 4 hours. In long run, Beverage Additive could be helpful in preventing alcohol induced degenerative disorders which are related with DNA damage. The present phase II clinical study indicates the probable contribution of Beverage Additive in concomitant protection of DNA damage as judged by comet assay and CBMN assay from peripheral lymphocytes and serum 80H-dG assay. All these findings suggested that Beverage Additive could be effective in reducing alcohol induced pathophysiological changes for a protracted period of time.

### Technical Advancements and Economic Significance:

The present invention provides the following technical advancements over prior art: An alcoholic beverage is provided which provides DNA protection.

### Definitions

As used in the present invention, the following words and phrases are generally intended to have the meaning as set forth below, except to the extent that the context in which they are used to indicate otherwise.

Active ingredient: The term active ingredient means a composite mixture of saponin glycoside and sugar or sugar alcohol, which alleviates oxidative stress, and resulting in DNA damage induced by xenobiotic such as alcohol.

Stress: Stress is a condition, which is triggered by generation of reactive oxygen species (“ROS”) or reactive nitrogen species (“RNS”) due to induced injury caused by xenobiotic like alcohol. This ultimately leads to imbalance and could cause oxidative stress

Saponin Glycoside: Includes components such as Glycyrrhizin, including a derivative or its isomer, such as 18α-Glycyrrhizin, 18β-Glycyrrhizin, 18α-mono ammonium glycyrrhizinate, 18β-mono ammonium glycyrrhizinate, or a combination thereof.

Sugar: Compounds such as D-Maltodextrin, L-Maltodextrin, D-Maltose, L-Maltose. D-Dextrose, L-Dextrose, D-Glucose, L-Glucose, D-Trehalose, L-Trehalose, D-Sucrose, L-Sucrose, D-Lactose, L-Lactose, Hydrogenated Starch Hydrolysates, D-Fructose, D-Galactose, or a mixture thereof.

Sugar alcohol: Compounds such as, D-Glycerol, L-Glycerol, D-Mannitol, L-Mannitol, D-erythritol, L-erythritol, D-xylitol, or L-xylitol, L-Maltitol, D-Maltitol, L-Sorbitol, D-Sorbitol, L-Lactitol, D-Lactitol, L-Isomalt, D-Isomalt or a mixture thereof.

Alcoholism: A chronic and often progressive disease that includes problems controlling drinking, being preoccupied with alcohol, continuing to use alcohol even when it causes problems, having to drink more to get the same effect (physical dependence), or having withdrawal symptoms when one rapidly decreases or stops drinking. (Definition adapted from Mayo Clinic Staff Definition. (“Diseases and Conditions: Alcoholism (Definition).” Mayo Clinic. (2014, Dec. 5). Retrieved May 11, 2015, from http://www.mayoclinic.org/diseases-conditions/alcoholism/basics/defin ition/con-20020866).

Alcoholic: An individual afflicted with alcoholism.

Moderate drink: Consumption of at least one but less than three alcoholic beverages a day for men, and at least one but less than two alcoholic beverages a day for women.

Binge drinking: Consumption by an individual of sufficient alcohol to raise the individual's blood alcohol content above 0.08%, which, for most adults, would be reached by consuming five drinks for men, or four drinks for women, during a two-hour period.

Synergistic Composition: An interaction of active ingredients which, when administered simultaneously, produce an overall biological effect greater than when administered individually, i.e., having biological effects greater than the sum of individual biological effects of any of them

DNA protection: The ability of alcohol to provide protection to the DNA from being damaged after consumption of the same.

Active sugar alcohol ingredient: A sugar alcohol such as D-Mannitol, D-Erythritol or L-Erythritol.

Active saponin glycoside ingredient: A saponin glycoside such as 18β-GA, 18α-GA, or a mixture with equal parts 18β-GA and 18α-GA.

1. A composition providing synergistic DNA protection comprising:
(a) saponin glycoside in a mass concentration range of 0.04% to 0.3%;
(b) sugar or sugar alcohol in a mass concentration range of 0.5% to 2.5%; and
(c) optionally, flavouring agents and pH adjusting agents;
wherein the said saponin glycoside comprises
(i) Glycyrrhizin (GA) selected from 18β-Glycyrrhizin or 18α-Glycyrrhizin, or a combination thereof; or
(ii) Glycyrrhizin salt (GA salt) selected from 18α-Glycyrrhizin mono ammonium salt or 18β-Glycyrrhizin mono ammonium salt, or a combination thereof; or
(iii) a combination thereof
wherein the composition effectively reduces alcohol-induced DNA damage, repairs damaged DNA and enhances the repair thereof.

2. The composition according to claim 1, wherein the saponin glycoside comprises Glycyrrhizin (GA) or Glycyrrhizin salt (GA salt), or a combination thereof.

3. (canceled)

4. (canceled)

5. The composition according to claim 1, wherein the Glycyrrhizin (GA) comprises equal parts of 18β-Glycyrrhizin and 18α-Glycyrrhizin.

6. The composition according to claim 2, wherein the saponin glycoside is in a mass concentration range of 0.04% to 0.3%.

7. The composition according to claim 1, wherein the sugar is selected from the group consisting of D-Maltodextrin, L-Maltodextrin, D-Maltose, L-Maltose, D-Dextrose, L-Dextrose, D-Glucose, L-Glucose, D-Trehalose, L-Trehalose, D-Sucrose, L-Sucrose, D-Lactose, L-Lactose, Hydrogenated Starch Hydrolysates, D-Fructose or D-Galactose, or a mixture thereof.

8. The composition according to claim 1, wherein the sugar alcohol is selected from the group consisting of D-Glycerol, L-Glycerol, D-Mannitol, L-Mannitol, D-Erythritol, L-Erythritol, D-Xylitol, L-Xylitol, L-Maltitol, D-Maltitol, L-Sorbitol, D-Sorbitol, L-Lactitol, D-Lactitol, L-Isomalt or D-Isomalt, or a mixture thereof.

9. The composition according to claim 1, wherein the sugar or sugar alcohol is in a mass concentration range of 0.5% to 2.5%.

10. The composition according to claim 6, wherein the sugar alcohol is D-Mannitol or L-Mannitol, or mixture of D-Mannitol and L-Mannitol.

11. The composition according to claim 6, wherein the sugar alcohol is D-Erythritol or L-Erythritol, or mixture of D-Erythritol and L-Erythritol.

12. The composition according to claim 1, wherein the pH adjusting agent is selected from the group consisting of potassium sorbate, monobasic sodium phosphate, dibasic sodium phosphate and tribasic sodium phosphate.

13. The composition according to claim 12, wherein the pH adjusting agent is potassium sorbate in a mass concentration range of 0.01% to 0.2%.

14. The composition according to claim 1, wherein the flavouring agent is selected from the group consisting of extracts of herbs, spices, fruit, and artificial flavour, in mass concentration range of 0.01% to 0.2%.

15. The composition according to claim 1, wherein the composition further comprises a combination of active ingredients selected from the group consisting of:
an active sugar alcohol ingredient in a mass concentration of 0.8% and an active saponin glycoside ingredient in a mass concentration of 0.04%;
an active sugar alcohol ingredient in a mass concentration of 1.2% and an active saponin glycoside in a mass concentration of 0.04%;
an active sugar alcohol ingredient in a mass concentration of 2.5% and an active saponin glycoside in a mass concentration of 0.04%;
an active sugar alcohol ingredient in a mass concentration of 0.8% and an active saponin glycoside in a mass concentration of 0.1%;
an active sugar alcohol ingredient in a mass concentration of 1.2% and an active saponin glycoside in a mass concentration of 0.1%;
an active sugar alcohol ingredient in a mass concentration of 2.5% and an active saponin glycoside in a mass concentration of 0.1%;
an active sugar alcohol ingredient in a mass concentration of 0.5% and an active saponin glycoside in a mass concentration of 0.3%;
an active sugar alcohol ingredient in a mass concentration of 0.8% and an active saponin glycoside in a mass concentration of 0.3%; and
an active sugar alcohol ingredient in a mass concentration of 1.2% and an active saponin glycoside in a mass concentration of 0.3%.

16. The composition according to claim 1, wherein the composition is formulated as a beverage additive in a suitable physical form selected from liquid, solid, semi-solid, powder or viscous solid.

17. The composition according to claim 14, wherein the beverage additive is a blend of active ingredients, optionally a pH adjusting agent and a flavouring agent formulated as a solution with a solvent selected from distilled alcohol, deionized water or a combination of deionized water and distilled alcohol.

18. The composition according to claim 15, wherein said beverage additive is to be mixed with a beverage selected from the group consisting of alcoholic beverage and soft-drink beverage.

19. The composition according to claim 1 wherein the composition is an alcoholic beverage.

20. A synergistic beverage composition for protection of DNA damage comprising:
(a) saponin glycoside comprising 18β-glycyrrhizin, 18α-glycyrrhizin, or a combination thereof, in a mass concentration range of 0.04% to 0.3%;
(b) sugar alcohol comprising mannitol, xylitol or erythritol in a mass concentration range of 0.5% to 2.5%;
(c) quantum sufficit distilled alcohol or a combination of deionized water and distilled alcohol;
(d) optionally, a pH adjusting agent in a mass concentration range of 0.01% to 0.2%, and
(e) optionally, a flavouring agent in a mass concentration range of 0.01% to 0.2%;
wherein the combination of active ingredients in the composition at appropriate quantity/concentration of each active ingredients synergistically reduces DNA damage, repairs damaged DNA and enhances repair thereof.

21. The synergistic beverage composition according to claim 20, wherein the composition further comprises a combination of active ingredients selected from the group consisting of:
an active sugar alcohol ingredient in a mass concentration of 0.8% and an active saponin glycoside ingredient in a mass concentration of 0.04%;
an active sugar alcohol ingredient in a mass concentration of 1.2% and an active saponin glycoside in a mass concentration of 0.04%;
an active sugar alcohol ingredient in a mass concentration of 2.5% and an active saponin glycoside in a mass concentration of 0.04%;
an active sugar alcohol ingredient in a mass concentration of 0.8% and an active saponin glycoside in a mass concentration of 0.1%;
an active sugar alcohol ingredient in a mass concentration of 1.2% and an active saponin glycoside in a mass concentration of 0.1%;
an active sugar alcohol ingredient in a mass concentration of 2.5% and an active saponin glycoside in a mass concentration of 0.1%;
an active sugar alcohol ingredient in a mass concentration of 0.5% and an active saponin glycoside in a mass concentration of 0.3%;
an active sugar alcohol ingredient in a mass concentration of 0.8% and an active saponin glycoside in a mass concentration of 0.3%; and
an active sugar alcohol ingredient in a mass concentration of 1.2% and an active saponin glycoside in a mass concentration of 0.3%.
